(19) United States
(12) Patent Application Publication
(10) Pub. No.: US 2009/0136590 A1
(43) Pub. Date: May 28, 2009

(54) ANTI-TUMORAL, ANTIBACTERIAL AND ANTIVIRAL PHARMACEUTICAL COMPOSITION (VARIANTS)

(75) Inventor: Vladimir Mikhaylovich PLOTNIKOV, Tomsk (RU)

Correspondence Address: HOUSTON ELISEEVA, 4 MILITIA DRIVE, SUITE 4, LEXINGTON, MA, 02421, US

(73) Assignee: Nikolay Aleksandrovich BADULIN, Moscow (RU)

(21) Appl. No.: 12/346,172

(22) Filed: Dec. 30, 2008

Related U.S. Application Data
(63) Continuation of application No. PCT/RU2007/000341, filed on Jun. 21, 2007.

(30) Foreign Application Priority Data
Jul. 12, 2006 (RU) 2006125085

(51) Int. Cl. A61K 33/38

(52) U.S. Cl. 424/618

(57) ABSTRACT

The inventive pharmaceutical compositions relate to medicine and veterinary sciences, in particular to agents which exhibit a combined anti-tumoral, antibacterial and antiviral activity and can be used for producing medicinal and veterinary formulations. In the first variant, the composition contains in mass %: 0.016-1.6 silver nitrate, 0.07-0.7 alpha-asparaginic acid, 0.07-0.7 nicotinic acid, 0.03-1.4 hexamethylenetetramine, 0.02-5.0 sodium thiosulphate, the rest being water. In the second variant, the composition contains in mass %: 0.016-1.6 silver nitrate, 0.03-1.4 hexamethylenetetramine or imidazol, 0.056-5.6 cis-[Pt(NH₃)₂Im₂]Cl₂ or cis-[Pt(NH₂OH)₂Im₂]Cl₂, 0.07-0.7 alpha-asparagic acid or asparagin, 0.07-0.7 nicotinic acid, the rest being water. Said pharmaceutical compositions exhibit the anti-tumoral activity, which is greater than that of the known agent, and simultaneously perform antibacterial and antiviral action on an organism.

### RELATED APPLICATIONS

This application is a Continuation of PCT application serial number PCT/RU2007/000341, filed on Jun. 21, 2007, which claims priority to Russian Patent Application No. RU 2006125085, filed on Jul. 12, 2006, both of which are incorporated herein by reference in their entirety.

### FIELD OF THE INVENTION

Proposed pharmaceutical composition pertains to fields of medicine/medical science and veterinary science, specifically to the class of agents with combined anti-tumoral, antibacterial and antiviral activity; composition may be used in manufacturing of medical and veterinary pharmaceuticals.

### BACKGROUND OF THE INVENTION

A known anti-tumoral and antibacterial pharmaceutical composition includes silver-containing compounds such as silver nitrate; hexamethylenetetramine and tetra-iodine-hexamethylenetetramine (Pat. RU 2181050, MΠK7 A61 K33/18, A61 K33/38, 2002). The above composition is, however, quite toxic with poor water solubility, and thus, cannot be utilized in pharmaceutical preparations intended for internal use.

Other known anti-tumoral compositions are based on platinum (Pat. RU 2074861, Mar. 10, 1997 MΠK 6 C07F15/00) and its compounds including platinum cis-amine-di-imidazolyl dichloride derivatives, with anti-proliferative and cytostatic activity (Pat. RU 2114858, MΠK6 C07F15/00, A61K31/295, 1998). These compound also are quite toxic and do not have a complex effect as they to not exhibit significant enough antibacterial properties.

A composition that most closely fulfills the objectives of the proposed composition in combination of its properties and achievement of the desired effect is an antiviral and cytostatic agent with a following composition: (% mass)—hexamethylenetetramine, or imidazole, or antipyrin (phenazone)—0.007-0.70: silver nitrate—0.0085-0.85; sodium thiosulfate—0.0284-2.84; water or saline—to 100 (Pat. RU 2094048, 1997).

Due to use of silver salts as cytostatic agents, the proposed prototype pharmaceutical composition is a more progressive tumor treatment, however, its efficiency is limited by its toxicity to living organisms. It is worth noting, that bacterial infections often accompany malignant tumors, leukemias and lymphomas due to decrease of the organism's immune function, brought on in part by chemotherapy agents. Furthermore, production of antipyrin (phenazone) is presently discontinued, as more effective components are now available.

### SUMMARY OF THE INVENTION

Main objective of the described group of inventions is the development of pharmaceutical compositions with a higher anti-tumoral activity (compared to existing treatments) that also exhibit significant antibacterial and antiviral properties. Decrease in biological toxicity is a further effect of the proposed invention.

Above objective is achieved by supplementing the existing composition base (containing a silver salt, hexamethylenetetramine, sodium thiosulfate and water) with alpha-aspartic acid (or asparagine) and nicotinic acid, at (% mass):

| | |
|---|---|
| Silver nitrate | 0.016-1.6 |
| Hexamethylenetetramine | 0.03-1.4 |
| Sodium thiosulfate | 0.02-5.0 |
| Alpha-aspartic acid (asparagine) | 0.07-0.7 |
| Nicotinic acid | 0.07-0.7 |
| Water | to 100 |

Another approach to achieving the main objective is introduction of the imidazole-containing platinum compounds to the composition, such as cis-[Pt(NH₃)₂Im₂]Cl₂ or cis-[PtNH₂(OH)₂Im₂]Cl₂ at (% mass):

| | |
|---|---|
| Silver nitrate | 0.016-1.6 |
| Hexamethylenetetramine or imidazole | 0.03-1.4 |
| cis-[Pt(NH₃)₂Im₂]Cl₂ or cis-[PtNH₂(OH)₂Im₂]Cl₂ | 0.056-5.6 |
| Alpha-aspartic acid (asparagine) | 0.07-0.7 |
| Nicotinic acid | 0.07-0.7 |
| Water | to 100 |

Achieving the main objective by including platinum compound would raise the treatment's costs; however, it improves the treatment's anti-tumoral activity while decreasing its toxicity and, therefore, is preferable for its medicinal value.

The increase of silver nitrate (main anti-tumoral and antibacterial agent) content in the range of 0.016-1.6 in the proposed composition does not lead to an increase in toxicity due to presence of hexamethylenetetramine alpha-aspartic acid (asparagine) and nicotinic acid (respective amounts denoted above). The mentioned compounds have a synergistic effect, lowering the toxicity of silver nitrate; in the first group (type 1) of proposed compositions this effect is exhibited by sodium thiosulfate and in the second group (type 2)—by hexamethylenetetramine or imidazole.

Presence of platinum compounds in the second group of proposed compositions (type 2) improves the treatment's anti-tumoral activity while decreasing its toxicity and, therefore, is preferable for medical use. Proposed compositions in the first group (type 1) are preferable for veterinary used due to their lower cost.

Literature analysis demonstrated that there are no existing treatments analogous to the proposed composition in sum of its therapeutic properties; therefore, the proposed invention is patentable as “novel”.

A search for known technologies in related areas did not reveal any existing technology with a set of unique attributes completely analogous to that of the proposed invention. Since the significance of the unique characteristics for the desired effect has not been shown, this invention is meets patentability requirement “non-obvious”.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 illustrates dependency of tumor cell proliferation on treatment with proposed pharmaceutical composition in minimal component amounts, as compared to the prototype.

FIG. 2 illustrates dependency of tumor cell proliferation on treatment with proposed pharmaceutical composition in optimal component amounts, as compared to the prototype.

FIG. 3 illustrates dependency of tumor cell proliferation on treatment with proposed pharmaceutical composition in maximal component amounts, as compared to the prototype.

### DETAILED DESCRIPTION OF THE PREFERRED EMBODIMENTS

In the following examples an aqueous solution of the pharmaceutical composition was used with varying amounts of its known and novel components.

Table 1 shows data demonstrating effect of the proposed pharmaceutical composition on tumor cell death, thus, verifying its anti-tumoral property. Table 2 shows data on the composition's anti-tumoral activity in model organisms. Table 3 contains data on antibacterial properties of the proposed pharmaceutical composition. Table 4 shows results of the toxicological studies comparing the proposed pharmaceutical composition (composition types 1 and 2) with an existing platinum-based cancer treatment—Cisplatin. Table 5 shows data on comparative analysis of antiviral properties of composition type 2 of the proposed pharmaceutical composition. Table 6 shows data on assaying the anti-tumoral activity of the proposed pharmaceutical composition, type 2.

### Example 1

0.016 g of silver nitrate is mixed with 0.03 g of hexamethylenetetramine and 0.02 g of sodium thiosulfate; 0.07 g of alpha-aspartic acid and 0.07 g of nicotinic acid are added (dissolved in appropriate volume of water). Note cloudiness of resulting solution. The composition is tested for its anti-tumoral, antibacterial and antiviral activity.

### Example 2

1 g of silver nitrate is mixed with 0.8 g of hexamethylenetetramine and 3 g of sodium thiosulfate; 0.4 g of nicotinic acid and 0.4 g of alpha-aspartic acid or asparagine are added (dissolved in appropriate volume of water). Resulting solution is tested for its anti-tumoral, antibacterial and antiviral activity.

### Example 3

1.6 g of silver nitrate is mixed with 1.4 g of hexamethylenetetramine and 5 g of sodium thiosulfate; 0.7 g of asparagine and 0.7 g of nicotinic acid are added (dissolved in appropriate volume of water). Reagents precipitate partially. The composition is tested for its anti-tumoral, antibacterial and antiviral activity.

Cytostatic anti-tumoral activity of the proposed pharmaceutical composition type 2 (with imidazole-containing platinum compounds) was assayed by ³H-thymidine incorporation into tumor cells after a 24-hour incubation. Change in radioactivity was measured using a Mark-III meter. Cytostatic activity was analyzed in human lymphoma cell lines K-562 and HuT-102 (grown in RPMI-1640 (Serva). 10% fetal calf serum, 2×10⁵ cells/ml) and in short-term cultures of Raucher virus induced lymphoma and Ehrlich carcinoma; assayed by ³H-thymidine incorporation and Trypan Blue staining after 24- and 48-hour incubation.

### Example 4

0.016 g of silver nitrate is mixed with 0.03 g hexamethylenetetramine: 0.056 g of cis-[Pt(NH₃)₂Im₂]Cl₂, 0.07 g alpha-aspartic acid or asparagine and 0.07 g of nicotinic acid are added (dissolved in appropriate volume of water). The composition is tested for its anti-tumoral, antibacterial and antiviral activity.

### Example 5

0.016 g of silver nitrate is mixed with 0.03 g imidazole; 0.056 g of cis-[Pt(NH₃)₂Im₂]Cl₂, 0.07 g alpha-aspartic acid or asparagine and 0.07 g of nicotinic acid are added (dissolved in appropriate volume of water). This represents minimal component amounts. The composition is tested for its anti-tumoral, antibacterial and antiviral activity.

### Example 6

0.016 g of silver nitrate is mixed with 0.07 g hexamethylenetetramine; 0.0056 g of cis-[PtNH₂(OH)₂Im₂]Cl₂ or cis-[Pt(NH₃)₂Im₂]Cl₂, 0.07 g alpha-aspartic acid or asparagine and 0.07 g of nicotinic acid are added (dissolved in appropriate volume of water). The composition is tested for its anti-tumoral, antibacterial and antiviral activity.

### Example 7

g of silver nitrate is mixed with 0.07 g imidazole; 5.6 g of cis-[PtNH₂(OH)₂Im₂]Cl₂ or cis-[Pt(NH₃)₂Im₂]Cl₂, 0.07 g alpha-aspartic acid or asparagine and 0.07 g of nicotinic acid are added (dissolved in appropriate volume of water). This represents maximal component amounts. The composition is tested for its anti-tumoral, antibacterial and antiviral activity.

### Example 8

1 g of silver nitrate is mixed with 0.8 g of hexamethylenetetramine, 4 g of cis-[PtNH₂(OH)₂Im₂]Cl₂ or cis-[Pt(NH₃)₂Im₂]Cl₂; 0.4 g alpha-aspartic acid or asparagine and 0.4 g of nicotinic acid are added (dissolved in appropriate volume of water). This represents optimal component amounts; using an optimal ratio of components within the proposed range produces a stable clear solution. The composition is tested for its anti-tumoral, antibacterial and antiviral activity.

### Example 9

In a prototype composition, 0.0085 g of silver nitrate is mixed with 0.007 g of hexamethylenetetramine (urotropine); 0.0284 g of sodium thiosulfate hydrate is added. Resulting clear solution is tested for its cytostatic and antiviral activity.

### Example 10

In a prototype composition, 0.085 g of silver nitrate is mixed with 0.07 g of hexamethylenetetramine; 0.284 g of sodium thiosulfate hydrate dissolved in water is added. Resulting clear solution is tested for its cytostatic and antiviral activity.

### Example 11

In a prototype composition, 0.85 g of silver nitrate is mixed with 0.7 g of hexamethylenetetramine; 2.84 g of sodium thiosulfate hydrate dissolved in water is added. Resulting clear solution is tested for its cytostatic and antiviral activity.

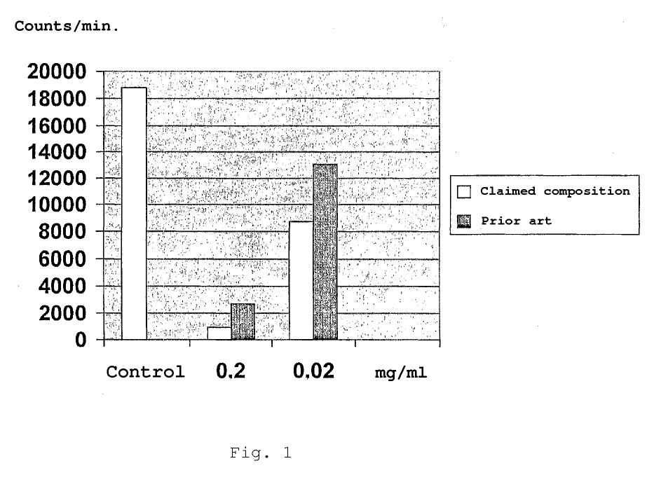

Experimental data supporting the importance of the proposed attributes for the invention's objective is presented in tables and in FIGS. 1-3.

Table 1 presents data on the effects of the proposed pharmaceutical composition on tumor cells. These demonstrate that the most effective composition (causing near complete suppression of the tumor cells) was that described in Example 2. The comparative data in Table 1 show that at the lower range boundary of the component amounts the prototype composition has very little effect on K-562 and HuT-102 cells.

A more detailed comparison of the proposed composition and prototype composition takes into account different anti-tumoral activity associated with different component amounts. It also proves achievement of the technical objective by the proposed composition in the entire proposed range of component amounts.

Table 2 summarizes data on anti-tumoral activity of the proposed composition in CBA mice with Ehrlich carcinoma (triple intra abdominal injection at 30 mg/kg). Treatment began 24 hours after introduction of tumor cells at 7×10⁶ cells/mouse. Treatment with proposed pharmaceutical composition increases lifespan by two fold compared to control (no treatment).

Table 2 characterizes in vivo anti-tumoral activity of the proposed composition type 1. According to Table 2. lifespan of control mice with Ehrlich carcinoma was 19.7 days; lifespan increased to 53.7 days with treatment with proposed composition type 1. It has been shown (Pat. RU 2094048) that treatment with prototype composition increases lifespan from 18.6 days (control) to 41.3 days (treatment). Thus, treatment with prototype composition increases specimens' lifespan by 2.22 fold, while treatment with the proposed composition in analogous experimental conditions increases lifespan by 2.73 fold.

Therefore, treatment with proposed composition increases lifespan of mice more than two-fold compared to untreated control, and has a higher anti-tumoral activity than the prototype treatment.

Antibacterial and Antiviral Activity:

Antibacterial activity of the proposed composition was assayed by the method of serial dilutions of the pharmaceutical composition in bacterial cultures, at concentrations of 1 mg/ml and 10 mg/ml. cis-[PtNH₂(OH)₂Im₂]Cl₂ or cis-[Pt(NH₃)₂Im₂]Cl₂ have analogous effects in the context of the experiments and both were used for assaying antiviral attributes of the proposed composition. Table 3 shows experimental results after microorganisms were incubated for 24 hours at 37° C. They demonstrate that proposed composition has a pronounced cytostatic effect on bacterial cells, showing 100% cell death at the concentrations tested (at optimal component amounts range—90%, at other amounts—50-70%).

Cultures of Raucher virus were treated with proposed composition at 1 mg/ml and 10 mg/ml for 4 hours. BALB/c mice were than inoculated with treated cultures. Treatment had a 100% protective effect against the virus on the animals (no deaths).

Studies of the optimal composition's (Examples 2 and 8) acute toxicity were done in BALB/c mice; aqueous composition solution was introduced by intra abdominal injection, in concentration range 15 mg/kg to 120 mg/kg (Table 4); animals were then observed for 30 days. Cisplatin, a known clinical treatment, was used for comparison. Toxicity of the proposed composition was shown to be significantly lower than that of Cisplatin.

Treatment with the proposed pharmaceutical composition inhibits occurrence of viral hemoblastosis in animals: treated group showed 8.5% lethality (5 of 35 animals) compared to 100% lethality in untreated control group (n=35).

Table 5 summarizes data on antiviral activity of the proposed composition type 2, as assayed by its ability to inhibit viral hemoblastosis, at minimal (Example 5), optimal (Example 8) and maximal (Example 7) component amounts. These data show that while the composition's antiviral activity is demonstrated throughout the range, percent lethality is lowest at the optimal component amounts.

Table 6 presents data on anti-tumoral activity of the proposed composition, tested in CBA mice with P815 mastocytoma treated with triple intra abdominal injection of the proposed composition at 30 mg/kg. Results demonstrate that treatment increases animals' lifespan by 3 fold compared to an untreated control.

Anti-tumoral cytostatic activity of platinum-containing compositions described in Examples 5, 8 and 7 with minimal, optimal and maximal component amounts is represented in FIGS. 1, 2 and 3, respectively. Dosages of 0.02 mg/ml and 0.2 mg/ml of the proposed composition were used and compared to same dosages of the prototype composition. The figures illustrate effects of the proposed pharmaceutical composition as assayed by ³H-thymidine incorporation by Raucher virus induced lymphoma cells after 24-hour incubation, compared to untreated control (impulses/minute, imp/min). Control consists of the tumor cell culture grown in the same conditions without treatment with platinum-containing composition; incorporation rate for control population was 18,750 imp/min. Figures show that the proposed composition (lighter bars) has a more pronounced inhibitory effect than that of the prototype composition (darker bars), allowing for a nearly complete inhibition of tumor cell proliferation. The anti-proliferative effect is dosage dependent, as results for different component amounts show (Examples 5, 7 and 8).

FIG. 1 shows that treatment with the proposed composition (as described in Example 5) and prototype composition (as describes in Example 9) produces a cytostatic effect as follows (imp/min): 0.2 mg/ml of proposed composition—930, prototype (same concentration)—2,700; 0.02 mg/ml of proposed composition—8,700, prototype (same concentration)—13,100.

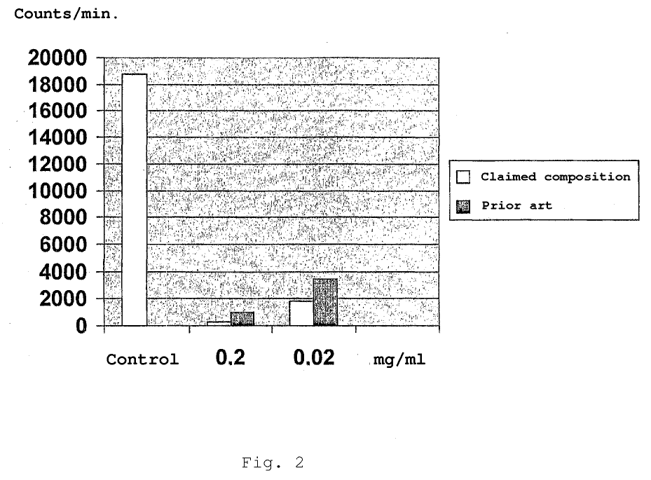

FIG. 2 shows a comparison for cytostatic activity of the proposed composition at optimal component amounts (Example 8) and prototype composition at comparable component amounts (Example 10); results for cytostatic activity are as follows (imp/min): 0.2 mg/ml of proposed composition—310, prototype (same concentration)—1,000; 0.02 mg/ml of proposed composition—1,770. prototype (same concentration)—3,480.

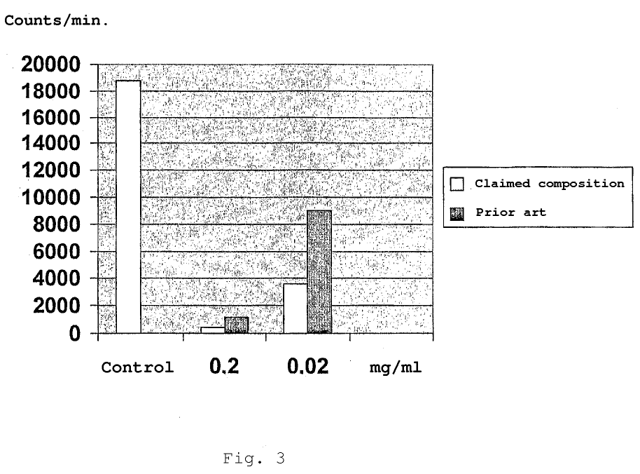

FIG. 3 shows a comparison for cytostatic activity of the proposed composition at maximal component amounts (Example 7) and prototype composition at comparable component amounts (Example 11); results for cytostatic activity are as follows (imp/min): 0.2 mg/ml of proposed composition—460, prototype (same concentration)—1,150; 0.02 mg/ml of proposed composition—3,600, prototype (same concentration)—9.000.

In an addition series of experiments, platinum-containing compositions were triple injected (30 mg/kg, intra abdominal injections) into 35 mice with Raucher virus induced lymphoma 24 hours after inoculation. The treatment suppressed occurrence of hemoblastosis, increasing survival rate to 90%; conversely, in untreated control group 100% lethality was observed.

### INDUSTRIAL FEASIBILITY

Proposed pharmaceutical composition (and its described variations) demonstrated high anti-tumoral activity as well as other complex effects in animals and cell culture. Note, that toxicity of the proposed pharmaceutical composition (and its described variations) is less pronounced than that of presently available treatments. This proposed pharmaceutical composition (and its described variations) can be used in pharmaceutical industry to manufacture clinical and veterinary treatments.

While this invention has been particularly shown and described with references to preferred embodiments thereof, it will be understood by those skilled in the art that various changes in form and details may be made therein without departing from the scope of the invention encompassed by the appended claims.

TABLE 1
Anti-tumoral cytostatic activity
of the proposed composition, in vitro
Cell death, %

| | |
|---|---|
| Proposed composition, mg/ml | Prototype composition, mg/ml |

| | Example 1 | | Example 2 | | Example 3 | | Example 9 | | Example 10 | | Example 11 | | Control |
|---|---|---|---|---|---|---|---|---|---|---|---|---|---|
| Cell type | 1 | 0.1 | 1 | 0.1 | 1 | 0.1 | 1 | 0.1 | 1 | 0.1 | 1 | 0.1 | — |
| K-562 | 65 | 30 | 100 | 100 | 100 | 70 | 29 | 15 | 90 | 65 | 85 | 60 | 8 |
| HUT-102 | 50 | 25 | 100 | 95 | 100 | 60 | 24 | 14 | 80 | 55 | 70 | 50 | 7 |

TABLE 2
Lifespan of mice with Ehrlich carcinoma (days)

| Proposed composition | | |
|---|---|---|
| (type I, Example 2) | Prototype composition | Control (untreated) |
| 53.7 ± 4.9 | 44.2 ± 3.1 | 19.7 ± 1.8 |

TABLE 3
Antibacterial activity of the proposed composition

| | | Bacterial growth inhibition, % | | | |
|---|---|---|---|---|---|
| Proposed | | | | | Mycobacterium |
| composition | mg/ml | Staphylococcus | Streptococcus | Escherichia coli | tuberculosis |
| Example 1 | 1 | 60 | 60 | 70 | 50 |
| | 10 | 100 | 100 | 100 | 100 |
| Example 2 | 1 | 100 | 100 | 100 | 100 |
| | 10 | 100 | 100 | 100 | 100 |
| Example 3 | 1 | 90 | 100 | 100 | 70 |
| | 10 | 100 | 100 | 100 | 100 |

TABLE 4
Toxicity of the proposed composition

| | Number of animals | | | Lethality, % | | |
|---|---|---|---|---|---|---|
| | Proposed | Proposed | | Proposed | Proposed | |
| Dosage, | composition | composition | | composition | composition | |
| mg/ml | (Ex. 1), no Pt | (Ex. 2), with Pt | Cisplatin | (Ex. 1), no Pt | (Ex. 2), with Pt | Cisplatin |
| 15 | 5 | 5 | 5 | 0 | 0 | 4/80% |
| 30 | 5 | 5 | 5 | 0 | 0 | 5/100% |
| 60 | 5 | 5 | 5 | 0 | 0 | 5/100% |
| 90 | 5 | 5 | 5 | 2/40% | 0 | 5/100% |
| 120 | 5 | 5 | 5 | 5/100% | 1/20% | 5/100% |

TABLE 5
Antiviral activity of the proposed composition (type 2)
assayed by inhibition of viral hemoblastosis
Lethality, %

| Proposed | Proposed | Proposed | |
|---|---|---|---|
| composition, | composition, | composition, | Control |
| Example 5 | Example 8 | Example 7 | (untreated) |
| 20 | 8.5 | 17.1 | 100 |

TABLE 6
Anti-tumoral activity of the proposed Pt-comtaining composition

| Tumor type | Proposed composition | Control (untreated) |
|---|---|---|
| P815 mastocytoma | 63.3 + 4.4 | 17.0 + 1.3 |

### WORKS CITED

1. Patent RU 2181050, MΠK 7 A61K33/18, A61K33/38, 2002. ANTITUMOR AND ANTIBACTERIAL COMPOSITION.
2. Patent RU 2074861, Mar. 10, 1997 MΠK 6 C07F 15/00. PLATINUM-CONTAINING PHARMACEUTICAL AGENTS.
3. Patent RU 2114858, MΠK 6 C07F15/00, A61K31/295, 1998. DERIVATIVES OF CIS-DIAMMINEDIIMIDAZOLYL PLATINUM DICHLORIDE SHOWING ANTIPROLIFERATIVE ACTIVITY AND A METHOD OF THEIR SYNTHESIS.
4. Patent RU 2094048, MΠK 6 A61K33/38, Oct. 27, 1991. CYTOSTATIC AND ANTIVIRAL COMPOSITION.

What is claimed is:

1. An anti-tumoral, antibacterial and antiviral pharmaceutical composition comprising silver nitrate, hexamethylenetetramine, sodium thiosulfate and water as a composition base, and alpha-aspartic acid (or asparagine) and nicotinic acid, at (% wt):

| | |
|---|---|
| Silver nitrate | 0.016-1.6 |
| Alpha-aspartic acid (asparagine) | 0.07-0.7 |
| Nicotinic acid | 0.07-0.7 |
| Hexamethylenetetramine | 0.03-1.4 |
| Sodium thiosulfate | 0.02-5.0 |
| Water | to 100 |

2. The anti-tumoral, antibacterial and antiviral pharmaceutical composition comprising silver nitrate, hexamethylenetetramine or imidazole and water as the composition base, further comprising imidazole-containing platinum compounds cis-[Pt(NH₃)₂Im₂]Cl₂ or cis-[PtNH₂(OH)₂Im₂]Cl₂, at (% wt):

| | |
|---|---|
| Silver nitrate | 0.016-1.6 |
| Hexamethylenetetramine or imidazole | 0.03-1.4 |
| cis-[Pt(NH3)2Im2]Cl2 or cis-[PtNH2(OH)2Im2]Cl2 | 0.056-5.6 |
| Alpha-aspartic acid (asparagine) | 0.07-0.7 |
| Nicotinic acid | 0.07-0.7 |
| Water | to 100 |
